(19) United States
(12) Patent Application Publication
(10) Pub. No.: US 2025/0279205 A1
(43) Pub. Date: Sep. 4, 2025

(54) SYSTEMS AND METHODS FOR VISUAL FIELD FORECASTING AND ESTIMATION

(71) Applicant: Oregon Health & Science University, Portland, OR (US)

(72) Inventors: Ashkan Abbasi, Portland, OR (US); Hiroshi Ishikawa, Portland, OR (US)

(21) Appl. No.: 19/069,203

(22) Filed: Mar. 3, 2025

Related U.S. Application Data
(60) Provisional application No. 63/560,579, filed on Mar. 1, 2024.

(51) Int. Cl. G16H 50/20

(52) U.S. Cl. CPC G16H 50/20

(57) ABSTRACT

Methods and systems for forecasting a patient's future pointwise visual field (VF) based on one or more visual field tests are disclosed. A hybrid deep learning framework is employed to combine the strengths of recurrent neural networks (RNN), convolutional neural networks (CNN), and transformers. Specific embodiments incorporate self-attention as part of a hybrid CNN and transformer architecture. The disclosed deep learning framework may also be used to generate an estimate of a patient's VF based on 2D or 3D optical coherence tomography (OCT) retinal image data provided as input.

### CROSS REFERENCE TO RELATED APPLICATION

The present application claims priority to U.S. Provisional Patent Application No. 63/560,579, file Mar. 1, 2024, the disclosure of which is hereby incorporated by reference.

### ACKNOWLEDGEMENT OF GOVERNMENT SUPPORT

This invention was made with government support under P30 EY010572, R01 EY013178, and R01 EY030929 awarded by The National Institutes of Health. The government has certain rights in the invention.

### TECHNICAL FIELD

This disclosure relates to systems and methods for visual field forecasting and visual field estimation to inform management of eye diseases such as glaucoma.

### BACKGROUND INFORMATION

Glaucoma is a leading cause of irreversible blindness that significantly impacts the quality of life of millions of people and poses a major public health concern as the global population ages. Accurate assessment of disease progression is essential for management of glaucoma and other eye diseases. A crucial component of glaucoma management is the longitudinal monitoring and assessment of patients' peripheral vision through visual field (VF) testing. In a VF test, a patient's responses to visual stimuli at different locations are recorded to measure the space in which the patient can see. Repeated VF testing over time allows clinicians to detect progressive loss of visual function. However, each VF testing session is tedious and time-consuming for the patient to complete, and the interpretation of results is somewhat subjective. Furthermore, VF testing is prone to high test-retest variability and progression monitoring requires long wait times between office visits to gather sufficient data to detect trends and forecast future vision loss. Conventional VF trend analysis methods require a minimum of three prior VF tests to obtain a rough estimate of the future VF. Considering the regular patient visit interval of 6 months, this means that the conventional methods require at least one and half years to make the first forecasting, which is not time and cost efficient. In a comprehensive study by Taketani et al. on the required number of prior VFs to precisely predict future VFs, they found that at least 10 prior VFs are needed to create a conventional regression model with an absolute prediction error of less than 2.5 decibels (dB), which will take 4.5 years of the patient follow-up period. Even with the slow progression nature of glaucoma, 4.5 years period is long enough to allow serious disease progression.

In recent years, deep learning-based methods have shown comparable performance to conventional trend analysis using only one prior VF test. However, their usage and accuracy are limited and, moreover, their forecasting performance can deteriorate when taking more than one VF as the input. In other words, these methods are able to capture population statistics for future VF forecasting, but they are unable to perform a true temporal and patient-specific analysis. Thus, there remains a need for better methods to more accurately forecast a patient's future VF using one or more prior VF tests as the basis for prediction.

### SUMMARY OF THE DISCLOSURE

Systems and methods for future visual field forecasting and visual field estimation are disclosed. The systems and methods described herein employ a hybrid approach that combines both spatial modeling and temporal modeling to deliver personalized forecasting with greater flexibility and improved performance using a few prior VF tests, and reduce the wait time between testing sessions needed for VF forecasting.

In some embodiments, the systems and methods comprise receiving or acquiring a most recent VF test, typically received in vector format, and its corresponding date of acquisition, and then forecasting or predicting the VF at a requested future date. The method of may entail normalization of the VF test data based on the range of values observed in a training data set, and includes converting the date of VF acquisition and the requested future date into scalar time displacement values which may also be normalized. VF and time displacement data are then reformatted into matrix format and passed through a trained 2-D convolutional neural network (2-D CNN) which provides a prediction of the VF at the requested future date. In some embodiments, the 2-D CNN may comprise a hybrid convolution and transformer architecture, and may incorporate a self-attention mechanism.

In other embodiments, the systems and methods disclosed herein may further be configured to also receive one or more prior VF tests and associated dates, the prior VF tests pre-dating the most recent VF test. When such prior VF test data is provided, they may be preprocessed and encoded (i.e., normalized and converted to time displacements) and input into a trained temporal processing module that encodes the temporal relationship among the preprocessed input and summarizes them into an intermediate representation. In some embodiments, the temporal processing module may be realized through a recurrent neural network (RNN) architecture. In some embodiment, this RNN architecture may comprise an RNN followed by a fully connected layer and a Gaussian Error Linear Unit (GeLU) activation function. Various RNN approaches may be implemented, including, but not limited to, for example long short-term memory (LSTM) or a gated recurrent unit ((GRU) architecture.

Further embodiments include a VF system configured to acquire VF data as part of testing performed during a patient visit, and/or retrieve VF test results from prior visits by that patient. Such a VF system may also include functionality to acquire or retrieve imaging data, such as optical coherence tomography (OCT) or OCT angiography data, for use in VF forecasting or VF estimation.

Additional aspects and advantages will be apparent from the following detailed description of preferred embodiments, which proceeds with reference to the accompanying drawings.

### BRIEF DESCRIPTION OF THE DRAWINGS

To easily identify the discussion of any particular element or act, the most significant digit or digits in a reference number refer to the figure number in which that element is first introduced.

FIG. 1 shows an example of a typical 24-2 Humphrey visual field test report, including a numerical sensitivity plot (left) and corresponding visual representation (right).

FIG. 2 shows an exemplary high-level flowchart for a method of forecasting visual field changes according to an embodiment described herein.

FIG. 3 shows a schematic diagram of a CNN architecture as described herein.

FIG. 4 is a plot showing mean absolute error (MAE) for different forecasting models used to predict VFs at increasingly distant future time-points.

FIG. 5 shows a VF estimation and forecasting results produced by different models.

FIG. 6 shows plots of pointwise mean absolute error (MAE) for the compared methods. For each method, the average MAE is also reported.

FIG. 7 shows line plots of errors (measured in MAE; the left vertical axis) for the compared methods against FNR (expressed as percentage ranges; the horizontal axis). The vertical bars represent the histogram of the number of samples, with the right vertical axis showing the sample count.

FIG. 8 shows line plots of errors (measured in MAE; the left vertical axis) of the VF forecasting methods against MD (the horizontal axis). The vertical bars represent the histogram of the number of samples, with the right vertical axis showing the sample count.

FIG. 9 is a plot showing the impact of varying the number of prior VF tests (the horizontal axis) used as the input on the average MAE (the vertical axis) for the compared VF forecasting methods.

FIG. 10 shows a set of examples of successful forecasting VF tests with different levels of vision loss and deficits using the Hybrid-VF-Net method disclosed herein.

FIG. 11 shows a set of examples of failed forecasting of VF tests using the Hybrid-VF-Net method disclosed herein.

FIG. 12 shows an example of how the disclosed Hybrid-VF-Net can be used gradually over time to forecast the future VF test using the two most recent prior VF tests.

FIG. 13 is a set of heatmaps showing the absolute differences between selected pairs of pointwise error plots (from FIG. 4). Larger magnitudes indicate locations where the second method—CascadeNet-5 in (a) and Hybrid-VF-Net in (b)—performs better than the other.

FIG. 14 schematically shows an example system for VF forecasting and estimation in accordance with the disclosure.

FIG. 15 schematically shows an example of a computing system in accordance with the disclosure.

### DETAILED DESCRIPTION OF EMBODIMENTS

The systems and methods disclosed herein address the problem of pointwise VF forecasting which is defined as forecasting VF test results at a given future time using prior VF test result(s). While recent deep learning methods have shown promising results for this problem, their practical use is still hindered by challenges such as limited modeling capacity, imbalanced datasets, and noisy labels. The inventors have noticed that prior methods either take a single prior VF (e.g., through spatial modeling with a convolutional neural network (CNN)) or multiple prior VFs (e.g., through temporal modeling using a recurrent neural network (RNN)). The present disclosure describes a hybrid deep learning framework that leverages the strengths of CNNs, transformers, and RNNs to enhance modeling capabilities, thereby improving both the flexibility and performance of forecasting.

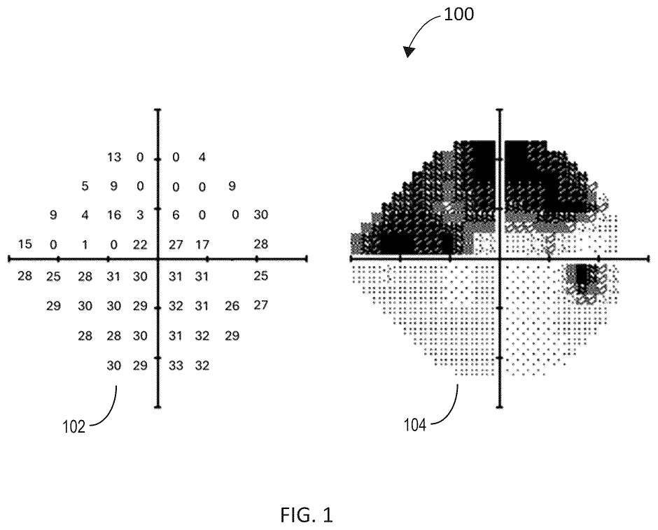

FIG. 1 shows a typical 24-2 Humphrey visual field test report which includes a numerical sensitivity plot 102 (left) and its corresponding grayscale map 104 (right) as a visual representation for easy interpretation. The numerical sensitivity plot is a grid of 52 test points (excluding the blind spots) that covers 24 degrees of the patient's visual field. The sensitivity at each test point is measured by presenting visual stimuli (i.e., lights of varying intensities) during the examination, with higher values indicating a greater ability to detect dimmer light, and zero representing an inability to detect even the brightest stimuli.

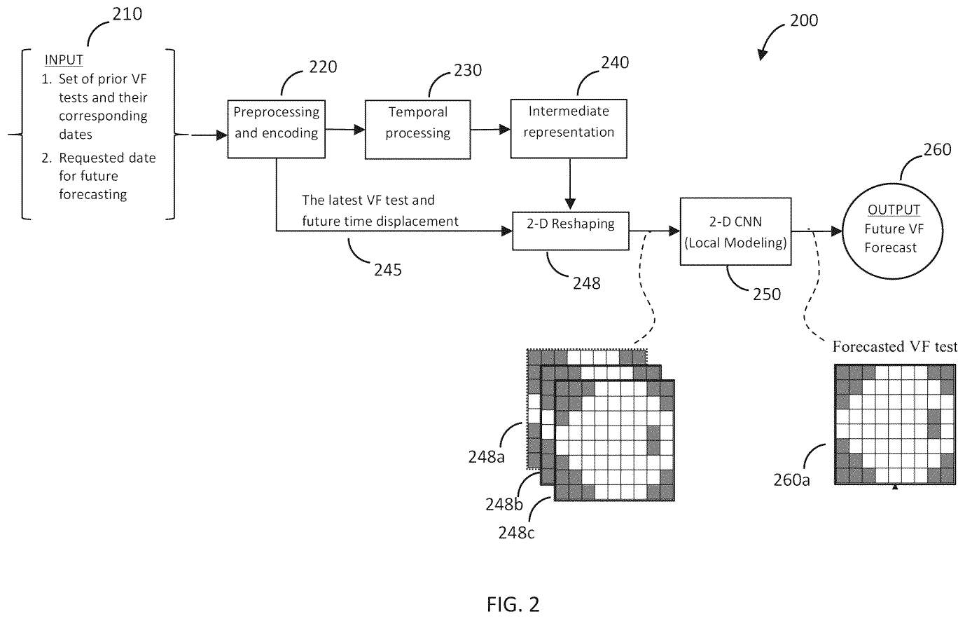

FIG. 2 shows an exemplary high-level flow diagram for a method 200 of forecasting a VF at a specified time in the future based on one or more prior VF tests and their corresponding dates of testing. Method 200 is configured to receive as input data 210 at least one ordered set of VF test sensitivity values represented in vector format (i.e., having vector length LVFT), the date on which each of the one or more set of VF test sensitivity values were acquired, and a future date for which a forecast or predicted VF is to be computed. By way of example, when a Humphrey Visual Field Analyzer, 24-2 SITA Standard is used to acquire patient VF test data, the VF test vector provided as input data 210 comprises LVFT=52 sensitivity values. Note that this set of input VF test sensitivity values are typically expressed in decibel (dB) units and exclude two (2) “blind spot” measurements from the 54 sensitivity values conventionally measured by the Humphrey Visual Field Analyzer.

A preprocessing and encoding step 220 receives input data 210 and normalizes the VF values of the test vector by dividing by a scalar value. In some embodiments, the scalar value used for normalization can be the maximum sensitivity value observed in the dataset used to train method 200. Preprocessing and encoding step 220 also converts the dates associated with VF tests and the future date for which a forecast is to be calculated to a time displacement. Each time displacement is defined by computing the number of days between the most recent VF test date and the others such that negative, zero, and positive numbers are assigned for the past, the most recent, and the requested (future) VF test dates, respectively. The time displacement values may also be normalized by dividing them by the maximum time displacement in the data set used to train method 200. Time displacements are appended to their corresponding VF test vectors, increasing each test vector length by one to LVFT+1. For example, in the case of visual field data acquired by Humphrey Visual Field Analyzer using the 24-2 SITA Standard, the length of the VF test vector would become 52+1. During this preprocessing and encoding step 220, the future VF vector is unknown and accordingly the unknown sensitivity values of the future VF test vector are filled with zeros and the normalized time displacement for the requested future date is appended to the zero-filled future VF vector.

After preprocessing and encoding step 220, if the number of input VF tests is greater than one, the data flow passes to a temporal processing module 230 that encodes the temporal relationship among the preprocessed input and summarizes them into an intermediate representation 240. Temporal processing module 230 may be implemented, for example, using a recurrent neural network (RNN) followed by a fully connected layer and a Gaussian Error Linear Unit (GeLU) activation function. The RNN of temporal processing module 230 may be implemented using various RNN architectures known in the art, such as long short-term memory (LSTM) or gated recurrent unit (GRU). In some embodiments, GRU may be implemented due to its simpler architecture and computational efficiency.

Intermediate output 240 as output by temporal processing module 230 is in the form of a VF test vector having vector length LVFT. A VF test vector is typically represented on a 2-D grid to interpret sensitivity variations across vision field (as depicted in FIG. 1). To align with this representation and make VF test vectors suitable for further processing, a 2-D reshaping module 248 reshapes intermediate output 240 into an 8×9 matrix (intermediate matrix representation) with extra locations padded with zeros (shaded matrix entries in FIG. 2). This 2-D reshaping module 248 also receives the latest VF test vector and the requested future time displacement and converts each of them into 2-D matrix representations having the same dimensions as zero-padded 2D matrix. Note that the future time displacement passed into module 248 is a single number that is transformed into a matrix by repeating this number to fill all non-zero padded matrix entries of the matrix. These three matrices—the latest VF, future time displacement, and intermediate representation matrices—are concatenated into channels to form an input tensor (248a, 248b, 248c in FIG. 2) to be passed into a 2-D convolutional neural network (2-D CNN) module 250 for local modeling. This arrangement preserves the structural integrity of the VF grid while enriching the input data for more robust forecasting.

In a preferred embodiment, module 250 is implemented as a 2-D CNN module with a self-attention mechanism, using a hybrid convolution and transformer architecture comprised of an inverted residual convolution layer, a relative self-attention block, and a fully connected layer. This architecture allows for both local and global processing of the module inputs. The final output from this model at 260 is a 2-D matrix 260 representing the forecasted VF sensitivity values.

Table 1 shows operational details for an exemplary embodiment of 2-D CNN with Attention Module 250 with exemplary matrix sizes to be passed as input/output between sequential operations. The residual convolution and transformer architecture embodied by module 250 is configured to perform the stepwise operations S0, S1, . . . , S9 as listed therein. The abbreviations used in Table 1 for various operations are as follows: CBG (convolution, batch normalization, and GeLU activation), MBConv (MobileNet-v2 convolution block), GAP (global average pooling), and MEAN (the average of the input along the first axis).

TABLE 1

| Steps | Operation | Output size |
|---|---|---|
| S0 | Input | 2 × 8 × 9 |
| S1 | Padding | 2 × 14 × 14 |
| S2 | 2 × CBG | 196 × 14 × 14 |
| S3 | 2 × MBConv | 392 × 14 × 14 |
| S4 | 2 × MBConv | 392 × 7 × 7 |
| S6 | 2 × Transformer | 196 × 2 × 2 |
| S7 | GAP | 196 × 1 × 1 |
| S8 | Reshape | 14 × 14 |
| S9 | MEAN(S1) + S8 | 14 × 14 |

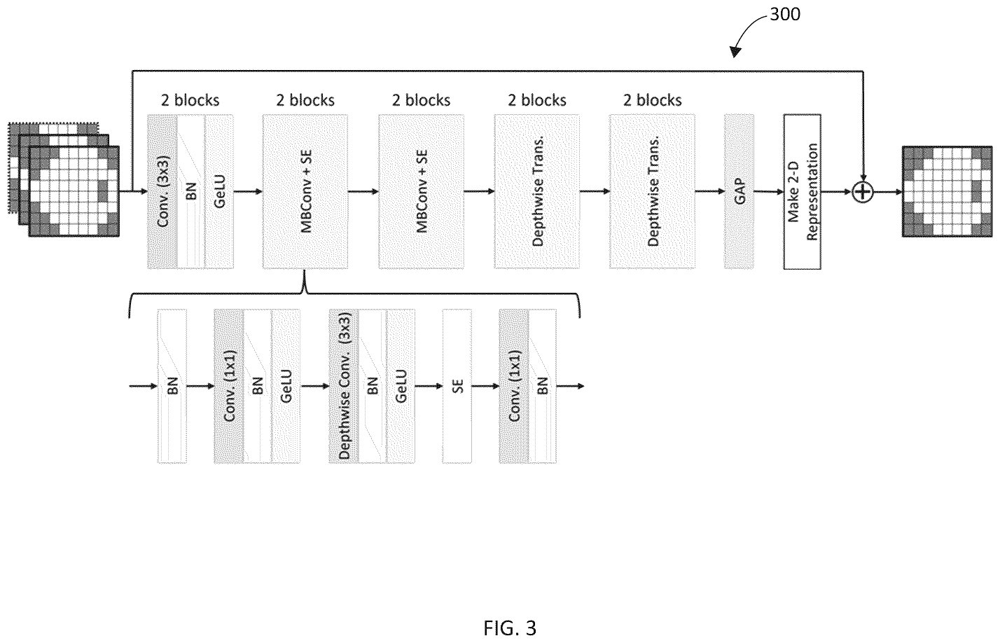

FIG. 3 further depicts schematically the CNN architecture 300 outlined in Table 1 used to implement the local modeling of module 250. In this CNN architecture 300, both convolution and depthwise transformer layers are used to implement a CNN with a self-attention mechanism equipped with a global residual connection. Additional description of CNN with self-attention can be found in (Dai Z, et. al., CoAtNet: Marrying Convolution and Attention for All Data Sizes. In: Ranzato M, Beygelzimer A, Dauphin Y, Liang P S, Vaughan J W, eds. Advances in Neural Information Processing Systems. Vol 34. Curran Associates, Inc.; 2021:3965-3977) and additional description of global residual connection can be found in (He K, et. al., Deep residual learning for image recognition. In: Proceedings of the IEEE Conference on Computer Vision and Pattern Recognition; 2016:770-778), both incorporated by reference herein. This design allows for both local and global processing of the input features. The global skip connection performs averaging across channels to match the dimension of input and output tensors. The abbreviations are as follows: Conv. (Convolution layer (kernel size)), BN (Batch normalization), GeLU (Gaussian error linear unit activation), MBConv. (MobileNet-v2 convolution layer), SE (Squeeze & Excitation), Depthwise Trans. (Depthwise transformer), and GAP (Global average pooling layer). Although not depicted in on the input and output datasets of FIG. 3, it can also be beneficial to symmetrically pad the input tensor before feeding it to local modelling module 250. This benefit to VF forecasting is likely due to the padding mitigating edge effects. Accordingly, in some embodiments, one may symmetrically pad a 3×8×9 input tensor to 3×14×14 before feeding it to the local modelling module 250, and then we remove the padding from the output tensor (1×14×14) and restore it to its original size (8×9) to obtain the forecasted VF sensitivity values.

Given a full training set of input and target (corresponding VF at specified future time) pairs, the architecture depicted in method 200 may be trained by minimizing MAE using optimization methods known in the art. In the examples presented in this disclosure, a stochastic optimization method called ADAM was run using 1000 epochs (see Kingma D, Ba J. Adam: A method for stochastic optimization. arXiv Prepr. Published online 2014). To realize the CNN depicted in FIG. 3, assuming an input with a spatial size 14×14, the first convolutional stage—which includes two blocks of convolutional layers, each one composed of a convolution followed by batch normalization and an activation—has 196 features. The sizes of subsequent feature maps are set to 392, 392, 784, and 196 for the remaining convolutional and depthwise transformer blocks, respectively.

### Example 1

Purpose: Accurate assessment of disease progression is essential for glaucoma management. The purpose of this study was to investigate how far in the future a deep learning model can forecast pointwise visual field (VF, Humphrey, 24-2 SITA Standard, Zeiss, Dublin, CA) data within the expected variability range based on one VF input.

Methods: We collected a series of VF test results in our longitudinal glaucoma cohort for each subject. A newly developed deep learning model architecture (called Hybrid-VF-Net in this example and described in detail above) which combines convolution and transformer (see “2-D CNN with attention” module 250 described above), was used to forecast pointwise VF sensitivities at variable future time points based on a single baseline VF data. Total number of 8390 VF series of 1423 subjects, where various intervals between VF tests were counted as individual series, were used for training, validation, and testing of the model. The time interval between the baseline and the forecasting future time point was concatenated with the baseline VF to form the input to the model. The mean absolute error (MAE) was used for both training and evaluation. As a comparison against the conventional architectures, CascadeNet-5 (convolutional neural network (CNN) architecture) and recurrent neural network (RNN, long short term memory (LSTM) architecture) were also trained. CascadeNet-5 took data from a single VF data as input as did Hybrid-VF-Net, while RNN took data from 2 consecutive baseline VF data as input. For all 3 models, the dataset was split to perform 10-fold cross-validation without patient overlap.

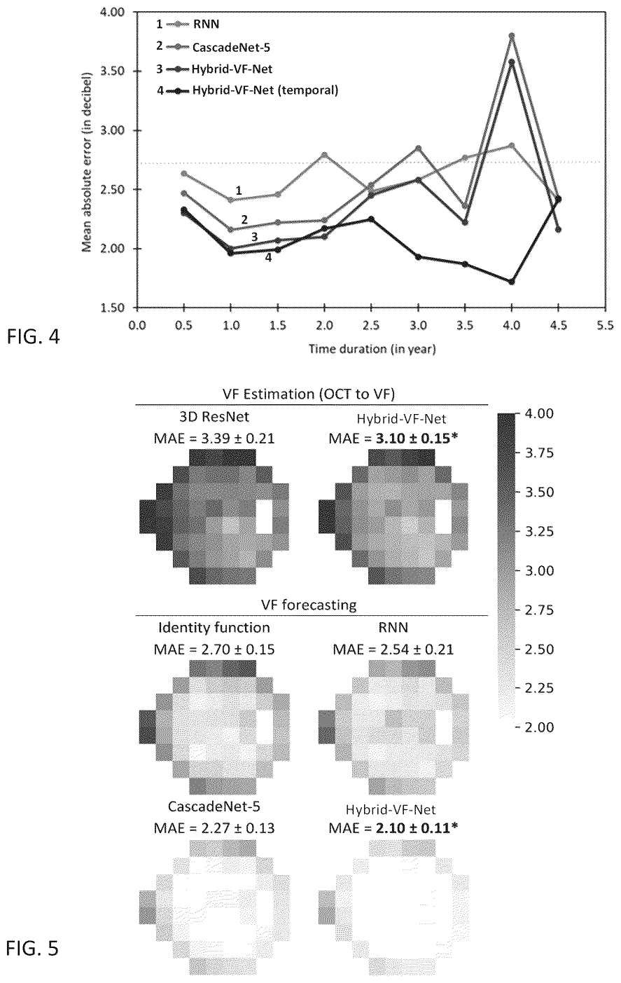

Results: Mean age of the subjects was 66.1±12.0 years. Average baseline VF mean deviation (MD) of the cohort was −5.6±7.4 dB (median −2.74, ranged from −34.0 to 5.8 dB). FIG. 4 shows performances of the 3 deep learning models as a function of time for forecasting. Hybrid-VF-Net showed the lowest MAE until it hit 4 years, all within the expected variability range (<2.75 dB), when both Hybrid-VF-Net and CascadeNet-5 showed a sudden rise in MAE, most likely due to small test samples.

Note that FIG. 4 also shows results from a fourth forecasting prediction (labeled “Hybrid-VF-Net (Temporal)”) that uses two VFs as input to the full model depicted in FIG. 2. By processing two VFs through the Hybrid-VF-Net network (i.e., using both module 230 and module 250), improved forecast performance is realized.

Conclusions: The newly developed Hybrid-VF-Net model achieved stable forecasting performance up to 3.5 years, and it outperformed the other tested models. Hybrid-VF-Net may provide a way to predict future glaucoma progression at the first visit, meaning that only one VF test is required.

### Example 2

Purpose: Different methods have been reported for visual field (VF) estimation from optical coherence tomography (OCT) or forecasting future VF using prior VFs. These methods are modality-specific and hard to compare with each other. Our goal is to test the efficacy of our unified framework to estimate and forecast VF using different input data (2D or 3D OCT, and VF).

Methods: From our longitudinal glaucoma cohort, we collected 8,390 pairs of 2 consecutive VFs (Humphrey, 24-2 SITA Standard, Zeiss, Dublin, CA) and their corresponding 3D OCT images (Cirrus HD-OCT, 200×200 ONH Scan, Zeiss). The average number of days between sessions was 342 days (range 90-2400). The dataset was split to perform 10-fold cross-validation without patient overlap. We utilized a hybridized convolution and transformer network (Hybrid-VF-Net) architecture (see Table 2 and Table 3; as well as the description for “2-D CNN with attention” module 250 above) comprising inverted residual convolution and transformer (relative self-attention and a fully connected layer) blocks to capture local and global patterns.

As shown in Table 2, in VF estimation for this Example 2, either a 2D (e.g. en face image, layer thickness map, etc.) or a down-sampled 3D OCT image can be used as input to Hybrid-VF-Net to estimate the corresponding VF (52 out of 54 values, excluding 2 blind spots). The abbreviations used for the operators are CBG (convolution, batch normalization, and GeLU activation), MBConv (MobileNet-v2 convolution block), and GAP (global average pooling).

As shown in Table 3, in VF forecasting for this Example 2, the input to Hybrid-VF-Net is made up of the current VF and the time difference (between the current and future VFs). This input is formed as a two-channel 8×9 matrix, where the first channel is filled with time difference value, and the second channel contains zero-padded VF sensitivity values). The abbreviations used for the operators are CBG (convolution, batch normalization, and GeLU activation), MBConv (MobileNet-v2 convolution block), and GAP (global average pooling).

Results: In VF estimation with en face images, 2D ResNet and Hybrid-VF-Net achieved the global mean absolute error (MAE) of 3.91±0.24, and 3.52±0.26 dBs, respectively. However, the overall performance was improved by using 3D OCT images. In FIG. 5, MAE and its pointwise heatmap are reported for the compared methods. It shows that 3D ResNet's error was 3.39±0.21 while Hybrid-VF-Net's error was 3.10±0.15. In VF forecasting, Hybrid-VF-Net (MAE=2.10±0.11) significantly outperformed the identity function (2.70±0.15), recurrent neural network (RNN) (2.54±0.21), and CascadeNet-5 (2.27±0.13) methods. Our analysis showed that VF forecasting performance stayed stable until the time interval hit 4 years.

Conclusions: The disclosed unified framework supported the use of 2D and 3D OCT, as well as VF, as inputs. The Hybrid-VF-Net model outperformed problem- and modality-specific methods for both VF estimation and forecasting by harnessing the power of local and global processing via the integration of convolutions and transformers in the network.

TABLE 2
VF Estimation Network: Enface or 3D OCT image to VF

| Operator Name | Output Size | Output size |
|---|---|---|
| Input | 1 × 200 × 200 | 1 × 72 × 144 × 72 |
| 2 × CBG | 54 × 100 × 100 | 54 × 36 × 72 × 36 |
| 2 × MBConv | 108 × 50 × 50 | 108 × 18 × 36 × 18 |
| 2 × MBConv | 218 × 25 × 25 | 218 × 9 × 18 × 9 |
| 2 × MBConv | 432 × 12 × 12 | 432 × 5 × 9 × 5 |
| 2 × MBConv | 432 × 6 × 6 | 432 × 3 × 5 × 3 |
| 2 × Transformer | 54 × 3 × 3 | 54 × 3 × 5 × 3 |
| GAP | 54 × 1 × 1 | 54 × 1 × 1 × 1 |
| Output | 54 | 54 |

TABLE 3
VF Forecasting Network (VF to next VF)

| Operator Name | Output Size |
|---|---|
| Input | 2 × 8 × 9 |
| Padding | 2 × 14 × 14 |
| 2 × CBG | 196 × 14 × 14 |
| 2 × MBConv | 392 × 14 × 14 |
| 2 × MBConv | 392 × 7 × 7 |
| 2 × Transformer | 784 × 4 × 4 |
| 2 × Transformer | 196 × 2 × 2 |
| GAP | 196 × 1 × 1 |
| Output | 14 × 14 |

### Example 3

In this Example 3, we present the experimental results for the proposed Hybrid-VF-Net method and compare its performance against two well-established VF forecasting methods: RNN (Park K, Kim J, Lee J. Visual field prediction using recurrent neural network. Sci Rep. Published online 2019; incorporated by reference herein) and CascadeNet-5 (Wen J C, Lee C S, Keane P A, et al. Forecasting future Humphrey Visual Fields using deep learning. Vavvas D G, cd. PLoS One. 2019; 14(4):1-1; incorporated by reference herein). To better analyze and demonstrate the effectiveness of the compared methods, we also define an identity method that always returns the most recent VF as the forecast for future VF. All the methods compared were re-implemented and we made our best efforts to set them up and train them optimally to achieve the best results. In the experiments described in this Example 3, unless otherwise stated, the identity and CascadeNet-5 methods take one prior VF test as input, while the RNN and the proposed Hybrid-VF-Net methods use two prior VF tests as input to forecast the future VF test result.

Dataset We gathered data from our longitudinal glaucoma cohort, which included 1,750 subjects—both healthy subjects and patients with glaucoma. The data were collected at the University of Pittsburgh and New York University according to the tenets of the Declaration of Helsinki for research involving human participants. The study was conducted in accordance with the regulations of the Health Insurance Portability and Accountability Act and was approved by the Institutional Review Board at each institution. Patients with best-corrected visual acuity of 20/60 or better and refractive error between −6.0 and +3.0 diopters were included, and those with a history of intraocular surgery or any ocular pathological conditions, other than glaucoma, that could affect OCT scanning, retinal layer thickness measurements, or both were excluded.

For each subject a series of Humphrey VF tests with the 24-2 Swedish Interactive Threshold Algorithm (SITA 24-2; Zeiss, Dublin, CA, USA) had been taken. The test intervals were at least 3 months, and the average number of days between visits was 342 days (range 90-2400). We excluded VF tests with more than 33% fixation losses or with false positive or false negative errors exceeding 15%. Our final dataset was comprised of 19,437 VF tests from 1,750 subjects. The average age at the first visit was 63.2±12.7 years, the number of VF visits per subject was 6.26±3.42, and the average mean deviation (MD) was −5.5±7.4.

To ensure robust comparisons, we partitioned the dataset for 5-fold cross-validation without patient overlap. In each fold, approximately 20% of the data was used for testing, while the remaining data was used for training and validation. We utilized the mean absolute error (MAE) as the main quantitative metric to evaluate the quality of the forecasts.

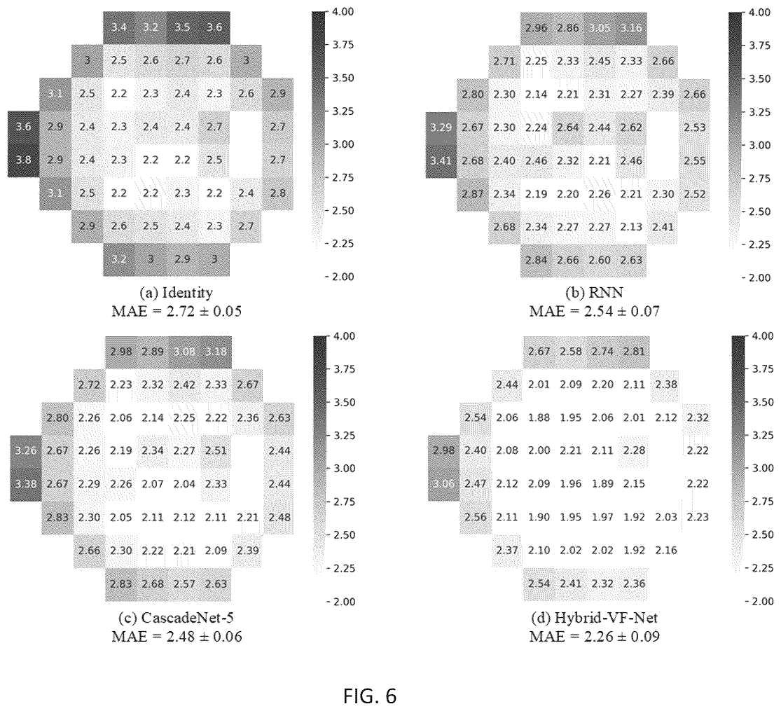

Error Comparisons FIG. 6 displays the average and pointwise MAEs for the compared methods. The pointwise error plot of the identity method (FIG. 6, panel (a)) shows that non-centered locations typically have the largest differences (between input and target values), which is expected due to the greater test-retest variability in VF test results at peripheral locations. Unsurprisingly, the error plots of other methods (FIG. 6, panels (b), (c), and (d)) also indicate higher errors at non-centered locations, suggesting that predictions in areas with higher variabilities are more challenging for all methods.

The pointwise plots in FIG. 6 panel (b) and FIG. 6 panel (c) show CascadeNet-5 (MAE=2.48) performed slightly better on average than RNN (MAE=2.54), though the overall difference is not substantial. However, the proposed Hybrid-VF-Net method (FIG. 6, panel (d)) surpassed RNN and CascadeNet-5 by combining temporal and local modeling approaches.

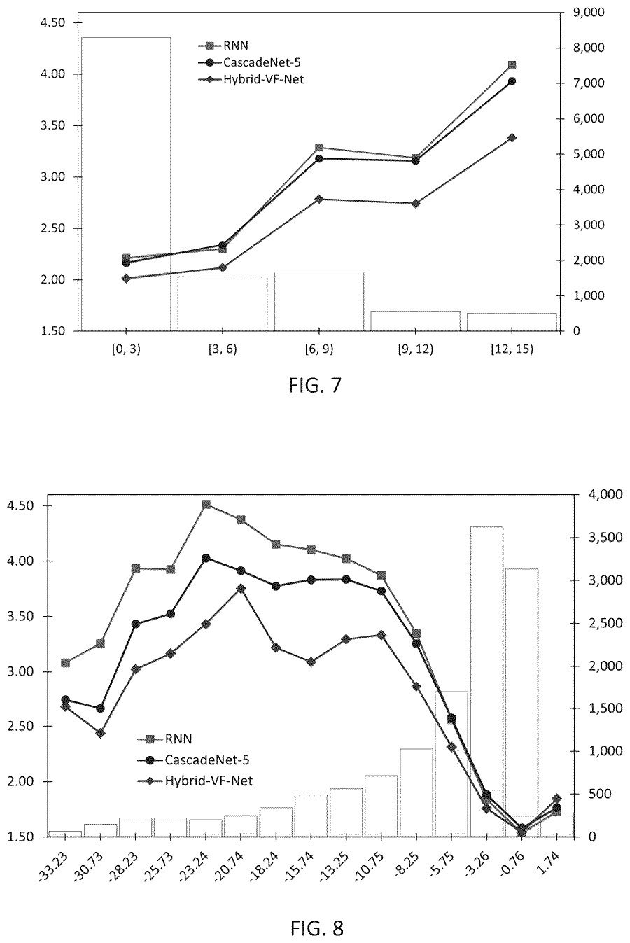

VF Test Reliability and Forecasting Performance The reliability of VF test results is generally assessed using three indices: fixation losses (FL), false positive rate (FPR), and false negative rate (FNR). Among these, FL has recently been reported to have minimal impact on reliability, whereas FPR and FNR are more influential in contributing to the test-retest variability of VF test results. More specifically, FNR is reported to significantly affect visual field assessment outcomes. Therefore, in FIG. 7, we demonstrated how variations in FNR influence the overall performance of the compared methods across different FNR ranges, with overlaid histograms showing the distribution of samples.

FIG. 7 demonstrates that when FNR is less than 6%, all the compared methods have relatively low MAEs (less than 2.5 dB), with RNN and CascadeNet-5 closely aligned, while Hybrid-VF-Net achieved a lower MAE. However, as FNR increases to [6, 9] percent range and beyond, the gap between the proposed Hybrid-VF-Net method and the other methods widens slightly, suggesting that Hybrid-VF-Net is more robust to increasing FNR. The overlaid bar plot illustrates the number of samples within each FNR range, clearly indicating that not only VF forecasting is more challenging when FNR increases, but also the number of samples decrease.

Forecasting Performance and Mean Deviation (MD) The MD measures the overall deviation of a patient's VF from the same age normative dataset of healthy subjects. The greater the VF loss, the lower MD value, which generally indicates greater glaucoma severity. In FIG. 8, we show how different VF forecasting methods perform in predicting VF changes across varying levels of disease severity.

FIG. 8 shows that overall, the proposed Hybrid-VF-Net demonstrated superior performance in maintaining lower errors for negative MD values (i.e., when VF is worse than the age-corrected average). However, for positive MD values (i.e., when VF is better than the age-corrected average), the proposed Hybrid-VF-Net method is not advantageous. For negative MD values, the RNN method shows higher error while CascadeNet-5 and the proposed Hybrid-VF-Net method are more stable 35. Additionally, this figure shows the distribution of samples (vertical bars), which is biased toward samples with mild vision loss (with MD values higher than −6 dB).

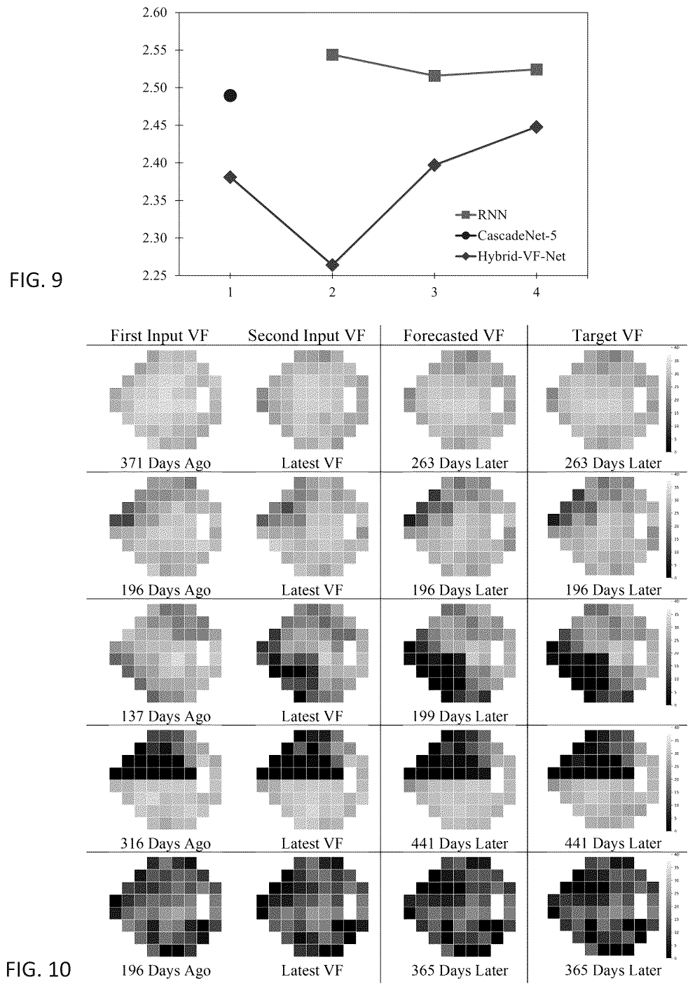

Effects of number of Prior VFs as Input While reducing the number of prior tests is desirable to decrease the long wait times required for VF forecasting, it is not always optimal. For example, using only one prior VF test as input, CascadeNet-5 has been reported better results than the trend-based regression and RNN methods. However, CascadeNet-5 has very limited patient-specific analysis capability due to its inability to incorporate past information. In FIG. 9, we investigate how altering the amount of prior information in the input affects the overall performance of VF forecasting methods.

FIG. 9 demonstrates that, although RNN, unlike CascadeNet-5, can incorporate past information, its performance changes slightly across different numbers of prior VF tests, and in all cases, the RNN's performance is below that of both CascadeNet-5 and the proposed Hybrid-VF-Net method. The best result for the RNN is achieved when using three prior VF tests, whereas the proposed Hybrid-VF-Net method consistently outperformed the RNN and CascadeNet-5 methods, regardless of the number of prior VF tests used as input. Notably, Hybrid-VF-Net excels particularly well in settings where only one or two prior VF tests are use.

VF Forecasting Examples Using Hybrid-VF-Net This section aims to qualitatively demonstrate usage examples of the proposed Hybrid-VF-Net method to forecast future VF tests. Three scenarios are considered to highlight the strengths and limitations of the proposed Hybrid-VF-Net method: success examples, failure examples, and VF test forecasting over time.

FIG. 10 presents some success examples, where the grayscale sensitivity heatmaps of the outputs closely resemble the target heatmaps. The examples have various levels of vision loss and VF deficit patterns. The first row shows VF tests of a relatively stable case with minimal vision loss. The second row depicts mild loss, while the third and fourth rows display signs of moderate vision loss. Additionally, the second and third rows are examples of rapid progressors (as considerable changes are evident within a relatively short period of time) with two different deficit patterns (superior and inferior quadrantopia deficits, respectively). The last row shows an example of profound vision loss.

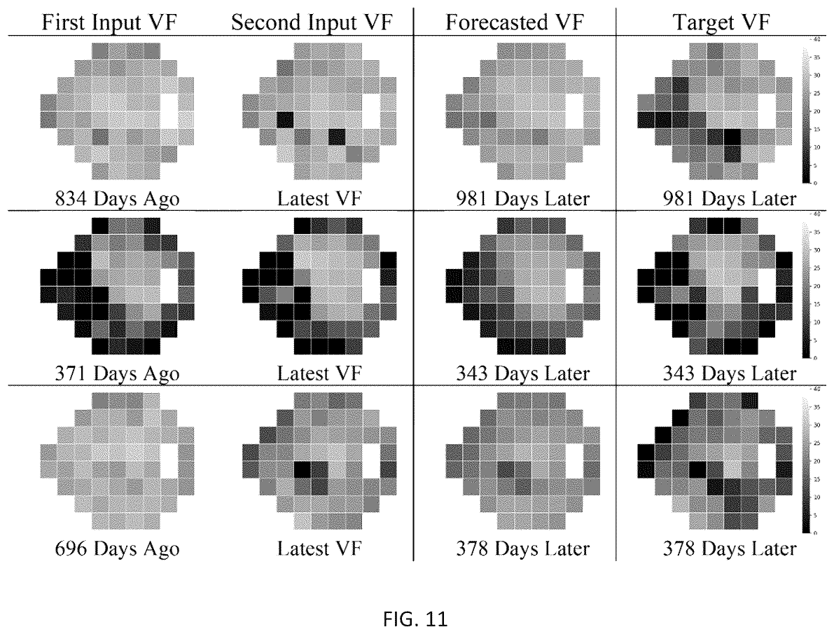

FIG. 11 presents some failure examples. In the first row, clearly the time intervals are so big. The method is asked to forecast the VF test of more than 2.5 years later using two inputs that are more than 2 years apart. In the second row, there is an inconsistency between the two inputs. For example, generally the second VF shows a considerably better VF test result than the first one. In the nasal part of the latest VF (just below the horizontal meridian), there are two bright locations that were previously dark in the first VF. Additionally, we noticed that the FNRs of these two VF tests are relatively high (14, and 12, respectively). The third row shows an example in which the two inputs are almost two years apart. Moreover, the FNRs for the inputs and target are 9, 13, 7, respectively.

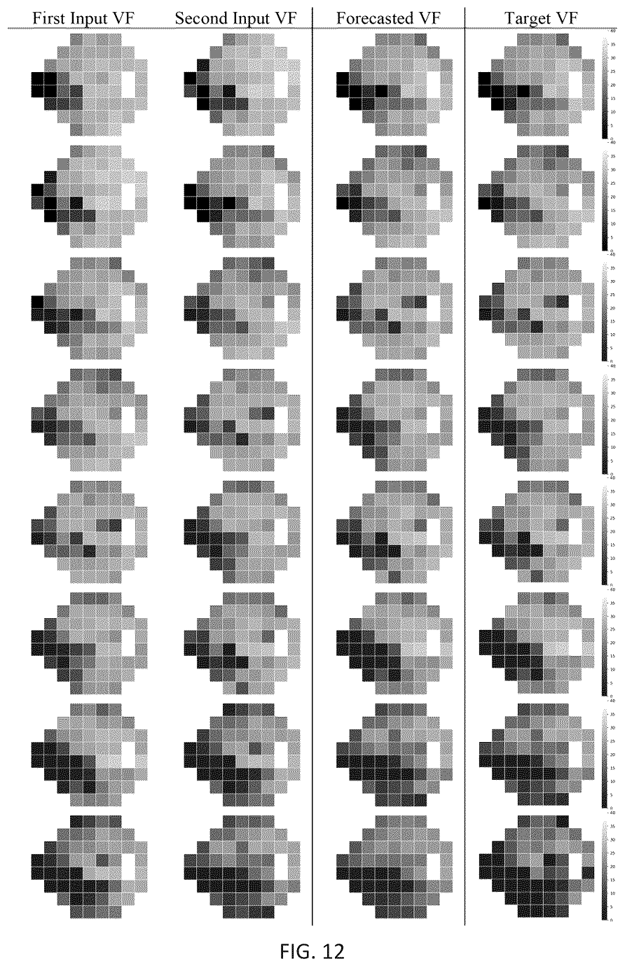

FIG. 12 illustrates how the proposed Hybrid-VF-Net method can be applied to forecast future VF tests gradually over time. With each new test result, the method can use the two most recent VF tests to forecast the next one. In the first row of the figure, the proposed Hybrid-VF-Net method took two prior VF tests and forecasted the next VF test. Then, in the subsequent rows, the second input VF and the target VF from the previous row are used as the two most recent inputs, and the method is tasked with predicting the future VF test.

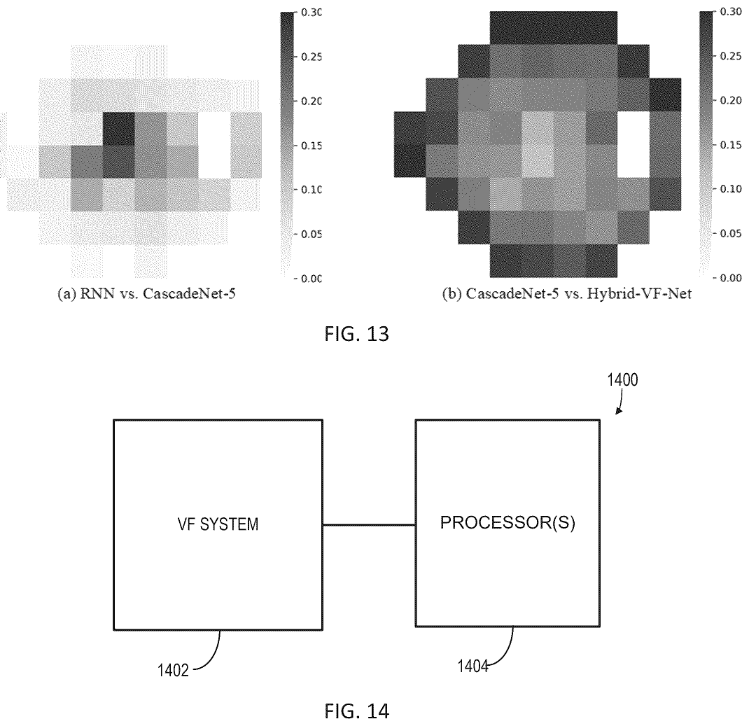

Discussion As noted above, we observed non-centered locations, which exhibit greater test-retest variability, also tend to have higher errors. To investigate further, in FIG. 13, we calculated the absolute differences between pairs of selected pointwise error plots (from FIG. 6) to pinpoint the specific locations where one method outperforms the other. FIG. 13, panel (a) shows that while CascadeNet-5 generally outperformed RNN, its improvements were mainly focused on centered locations. This is likely due to CascadeNet-5's local modeling approach, which leverages convolutional layers. In contrast, FIG. 13, panel (b) illustrates that the proposed Hybrid-VF-Net method outperformed CascadeNet-5 across both centered and non-centered locations, with particularly notable improvements in non-centered locations, which may be attributed to the integration of temporal modeling, as local modeling alone can provide limited clues for non-centered locations.

It is worth noting that our reimplementation of the RNN method used here differs from the method originally proposed by Park et al. in a few ways: First, rather than forecasting total deviations (TD), we trained the RNN to forecast raw sensitivity values. Second, we excluded VF test reliability indices (false negative rate, false positive rate, and fixation losses) from the input. Third, while the original RNN method used five prior VF tests as input, we only used two (except in the section “Effects of number of Prior VFs as Input” above). Although we did not investigate the role of including reliability indices as input, section “VF Test Reliability and Forecasting Performance” above provides a practical exploration of how using less reliable examples affects the performance of the compared methods.

In assessing VF test reliability and forecast performance, we observed that forecasting errors increased for all methods as input VF tests became less reliable. A key strength of the proposed Hybrid-VF-Net method is its greater resilience compared to the other methods. However, like many real-world medical datasets, our dataset is imbalanced, and the sample size decreases as reliability diminishes. This poses an inherent limitation on the experimental results, as the reduced number of samples negatively impacts the effectiveness of training and evaluation procedures. Nonetheless, an intriguing question is: which type of patients tend to have less reliable VF tests.

Higher FNRs are more common among patients with moderate to severe glaucoma because VF defects tend to increase test-retest variability. This fact aligns with our findings above: as the disease progresses (with MD becoming more negative, FIG. 7), errors across all methods follow a similar pattern-initially increasing, peaking in the range [−23.24, −18.24) dB, and then rapidly decreasing. This trend suggests that VF forecasting is more challenging in cases of moderate to severe glaucoma. Conversely, for patients with normal vision, mild vision loss, or profound vision loss, test-retest variability is lower, leading to lower VF forecasting errors (FIG. 8).

Another noteworthy observation is the impact of the number of prior VFs. While CascadeNet-5 can only use one prior VF as input, offering limited personalized forecasting capabilities, the RNN method is more flexible. However, the performance of the RNN method with respect to varying number of prior tests has not been studied before, probably because the authors' intention was to compare their results with an ordinary linear regression method, for which using fewer than five prior VF tests can lead to unstable results. Our findings suggest that the proposed Hybrid-VF-Net method can perform well with a limited prior VFs (one or two), which is advantageous in clinical settings where patients may have fewer available VF test results. However, FIG. 9 also shows that the performance of Hybrid-VF-Net is sensitive to the number of prior tests used as input. Ideally, adding more information should not lead to decreased performance. Therefore, we hypothesize that this performance drop may be due to limitations in the proposed framework (FIG. 2) in utilizing temporal information, though it is also worth noting that the RNN method shows a performance drop with four prior VF tests as well.

While promising VF forecasting examples using the proposed Hybrid-VF-Net method are illustrated above, a holistic analysis of both success and failure cases suggests that the severity of the disease, the quality of data, and the avoidance of using large time displacements critically influence the VF forecasting performance. Moreover, our previous quantitative analysis highlights several key challenges and opportunities: First, certain VF test locations are more difficult to forecast. Our experiments suggest that these locations particularly benefit from temporal modeling. Since they are mostly peripheral rather than central locations, local modeling is not beneficial. Second, forecasting error is not uniformly distributed across different disease severities, with moderate to advanced cases often presenting higher forecasting errors. Third, besides the severity of the disease, the datasets are also imbalanced in terms of data reliability. Data with lower reliability, especially in moderate to advanced glaucoma cases, negatively impact forecasting performance. This is further compounded by the difficulty in obtaining reliable VF tests in these cases. Therefore, targeted efforts toward acquiring additional moderate to advanced glaucoma cases may help alleviate dataset imbalances.

In conclusion, in the studies described above in this Example 3, we presented Hybrid-VF-Net, a hybrid deep learning model for visual field (VF) forecasting. Hybrid-VF-Net combines spatial and temporal modeling to enhance flexibility and accuracy compared to the existing deep learning-based VF forecasting methods. We conducted extensive experiments to demonstrate, compare, and analyze the performance of Hybrid-VF-Net, highlighting the merits and challenges of VF forecasting methods as well as the opportunities in applying deep learning to VF forecasting. The experimental results suggest that the proposed Hybrid-VF-Net method has greater resilience to the variability in data reliability and disease severity, enabling more reliable predictions even in challenging cases. This underscores its potentials for practical application in glaucoma management.

### Example 4

FIG. 14 schematically shows an exemplary system 1400 in accordance with various embodiments. System 1400 comprises a VF system 1402 configured to acquire new VF data or retrieve previously acquired VF data, and one or more processors or computing systems 1404 that are configured to implement the various processing routines described herein, including VF forecasting and VF estimation. VF system 1402 may comprise a VF analyzer such as, but not limited to, a Humphrey VF Analyzer and may further comprise other imaging modalities such as an optical coherence tomography system to acquire image data for use in VF estimation as described in Example 2 above.

In various embodiments, VF system 1402 may be adapted to allow an operator to perform various tasks. For example, a VF system may be adapted to allow an operator to configure and/or launch various ones of the herein described methods. In some embodiments, a VF system may be adapted to generate, or cause to be generated, reports of various information including, for example, reports of the results of VF analyses as exemplified in FIG. 1.

In embodiments of VF systems comprising a display device, data and/or other information may be displayed for an operator. In embodiments, a display device may be adapted to receive an input (e.g., by a touch screen, actuation of an icon, manipulation of an input device such as a joystick or knob, etc.) and the input may, in some cases, be communicated (actively and/or passively) to one or more processors. In various embodiments, data and/or information may be displayed, and an operator may input information in response thereto.

In some embodiments, the above described methods and processes may be tied to a computing system, including one or more computers. In particular, the methods and processes described herein, may be implemented as a computer application, computer service, computer API, computer library, and/or other computer program product.

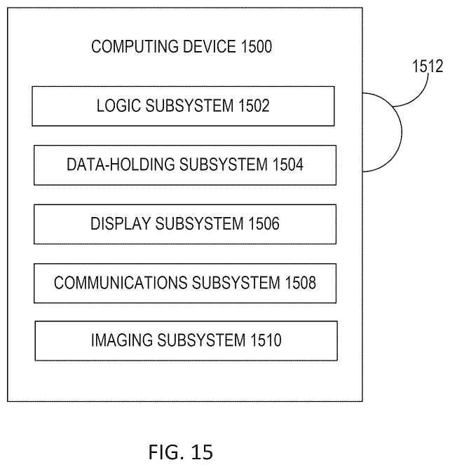

FIG. 15 schematically shows a non-limiting computing device 1500 that may perform one or more of the above described methods and processes. For example, computing device 1500 may represent a processor 1404 included in system 1400 described above, and may be operatively coupled to, in communication with, or included in an VF system (e.g., a VF testing and analysis device). Computing device 1500 is shown in simplified form. It is to be understood that virtually any computer architecture may be used without departing from the scope of this disclosure. In different embodiments, computing device 1500 may take the form of a microcomputer, an integrated computer circuit, printed circuit board (PCB), microchip, a mainframe computer, server computer, desktop computer, laptop computer, tablet computer, home entertainment computer, network computing device, mobile computing device, mobile communication device, gaming device, etc.

Computing device 1500 includes a logic subsystem 1502 and a data-holding subsystem 1504. Computing device 1500 may optionally include a display subsystem 1506, a communication subsystem 1508, an imaging subsystem 1510, and/or other components not shown in FIG. 15. Computing device 1500 may also optionally include user input devices such as manually actuated buttons, switches, keyboards, mice, game controllers, cameras, microphones, and/or touch screens, for example.

Logic subsystem 1502 may include one or more physical devices configured to execute one or more machine-readable instructions. For example, the logic subsystem may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

The logic subsystem may include one or more processors that are configured to execute software instructions. For example, the one or more processors may comprise physical circuitry programmed to perform various acts described herein. Additionally or alternatively, the logic subsystem may include one or more hardware or firmware logic machines configured to execute hardware or firmware instructions. Processors of the logic subsystem may be single core or multicore, and the programs executed thereon may be configured for parallel or distributed processing. The logic subsystem may optionally include individual components that are distributed throughout two or more devices, which may be remotely located and/or configured for coordinated processing. One or more aspects of the logic subsystem may be virtualized and executed by remotely accessible networked computing devices configured in a cloud computing configuration.

Data-holding subsystem 1504 may include one or more physical, non-transitory, devices configured to hold data and/or instructions executable by the logic subsystem to implement the herein described methods and processes. When such methods and processes are implemented, the state of data-holding subsystem 1504 may be transformed (e.g., to hold different data).

Data-holding subsystem 1504 may include removable media and/or built-in devices. Data-holding subsystem 1504 may include optical memory devices (e.g., CD, DVD, HD-DVD, Blu-Ray Disc, etc.), semiconductor memory devices (e.g., RAM, EPROM, EEPROM, etc.) and/or magnetic memory devices (e.g., hard disk drive, floppy disk drive, tape drive, MRAM, etc.), among others. Data-holding subsystem 1504 may include devices with one or more of the following characteristics: volatile, nonvolatile, dynamic, static, read/write, read-only, random access, sequential access, location addressable, file addressable, and content addressable. In some embodiments, logic subsystem 1502 and data-holding subsystem 1504 may be integrated into one or more common devices, such as an application specific integrated circuit or a system on a chip.

FIG. 15 also shows an aspect of the data-holding subsystem in the form of removable computer-readable storage media 1512, which may be used to store and/or transfer data and/or instructions executable to implement the herein described methods and processes. Removable computer-readable storage media 1512 may take the form of CDs, DVDs, HD-DVDs, Blu-Ray Discs, EEPROMs, flash memory cards, USB storage devices, and/or floppy disks, among others.

When included, display subsystem 1506 may be used to present a visual representation of data held by data-holding subsystem 1504. As the herein described methods and processes change the data held by the data-holding subsystem, and thus transform the state of the data-holding subsystem, the state of display subsystem 1506 may likewise be transformed to visually represent changes in the underlying data. Display subsystem 1506 may include one or more display devices utilizing virtually any type of technology. Such display devices may be combined with logic subsystem 1502 and/or data-holding subsystem 1504 in a shared enclosure, or such display devices may be peripheral display devices.

When included, communication subsystem 1508 may be configured to communicatively couple computing device 1500 with one or more other computing devices. Communication subsystem 1508 may include wired and/or wireless communication devices compatible with one or more different communication protocols. As non-limiting examples, the communication subsystem may be configured for communication via a wireless telephone network, a wireless local area network, a wired local area network, a wireless wide area network, a wired wide area network, etc. In some embodiments, the communication subsystem may allow computing device 1500 to send and/or receive messages to and/or from other devices via a network such as the Internet.

When included, imaging subsystem 1510 may be used acquire and/or process any suitable image data from various sensors or imaging devices in communication with computing device 1500. For example, imaging subsystem 1510 may be configured to acquire OCT image data, e.g., interferograms, 3D image data, or en face images, as part of an VF estimation system, e.g., VF system 1402 described above. Imaging subsystem 1510 may be combined with logic subsystem 1502 and/or data-holding subsystem 1504 in a shared enclosure, or such imaging subsystems may comprise periphery imaging devices. Data received from the imaging subsystem may be held by data-holding subsystem 1504 and/or removable computer-readable storage media 1512, for example.

Skilled persons will appreciate that many changes may be made to the details of the above-described embodiments without departing from the underlying principles of the invention. The scope of the present invention should, therefore, be determined only by claimed inventions and equivalents thereof.

What is claimed is:

1. A method for visual field (VF) forecasting, the method comprising:
receiving a most recent VF test vector, its corresponding most recent date of acquisition, and a requested future date;
submitting to a trained forecasting model the most recent VF test vector and its corresponding most recent date of acquisition; and
forecasting a future visual field at the requested forecast date based on the output of the trained forecasting model.

2. The method of claim 1, wherein forecasting the future visual field at the requested forecast date based on the output of the trained forecasting model comprises the steps of:
normalizing the most recent VF test vector;
converting and normalizing the most recent date of acquisition to generate a most recent time displacement;
converting and normalizing the requested future date to generate a future time displacement;
reshaping each of the normalized most recent VF test vector, the most recent time displacement, and the future time displacement into matrix format, and concatenating them into channels to form an input tensor; and
providing the input tensor as input to a trained 2-D convolutional neural network (2-D CNN), thereby generating the future visual field at the requested forecast date.

3. The method of claim 2, wherein the 2-D CNN comprises a hybrid convolution and transformer architecture including an inverted residual convolution layer, a relative self-attention block, and a fully connected layer.

4. A method for visual field (VF) forecasting, the method comprising:
receiving a most recent VF test vector, its corresponding most recent date of acquisition, and a requested future date;
receiving one or more prior VF test vectors and corresponding prior dates of acquisition, wherein the prior dates of acquisition are earlier than the most recent date of acquisition;
submitting to a trained forecasting model the most recent VF test vector and its corresponding most recent date of acquisition, along with the one or more prior VF test vectors and corresponding prior dates of acquisition; and
forecasting a future visual field at the requested forecast date based on the output of the trained forecasting model.

5. The method of claim 4, wherein forecasting the future visual field at the requested forecast date based on the output of the trained forecasting model comprises the steps of:
normalizing the most recent VF test vector;
converting and normalizing the most recent date of acquisition to generate a most recent time displacement;
converting and normalizing the requested future date to generate a future time displacement;
normalizing the one or more prior VF test vectors;
converting and normalizing the prior dates of acquisition to generate a set of prior time displacements;
providing the one or more normalized prior VF test vectors and set of prior time displacements, along with the normalized most recent VF test vector and the most recent time displacement as input to a temporal processing module to generate a set of intermediate temporal representation vectors;
reshaping each of the set of intermediate temporal representation vectors, the normalized most recent VF test vector, the most recent time displacement, and the future time displacement into matrix format, and concatenating them into channels to form an input tensor; and
providing the input tensor as input to a trained 2-D convolutional neural network (2-D CNN), thereby generating the future visual field at the requested forecast date.

6. The method of claim 5, wherein the 2-D CNN comprises a hybrid convolution and transformer architecture including an inverted residual convolution layer, a relative self-attention block, and a fully connected layer.

7. The method of claim 5, wherein the temporal processing module comprises a recurrent neural network (RNN) followed by a fully connected layer and a Gaussian Error Linear Unit (GeLU) activation function.

8. The method of claim 7, wherein the RNN is implemented using a long short-term memory (LSTM) architecture.

9. The method of claim 7, wherein the RNN is implemented using a gated recurrent unit (GRU) architecture.

10. A VF forecasting system, the system comprising:
a VF system;
a logic subsystem; and
a data holding subsystem comprising non-transitory machine-readable instructions stored thereon that are executable by the logic subsystem to perform the steps of:
receiving, via the VF system, a most recent VF test vector, its corresponding most recent date of acquisition, and a requested future date;
receiving, via the VF system, one or more prior VF test vectors and corresponding prior dates of acquisition, wherein the prior dates of acquisition are earlier than the most recent date of acquisition;
submitting to a trained forecasting model the most recent VF test vector and its corresponding most recent date of acquisition, along with the one or more prior VF test vectors and corresponding prior dates of acquisition; and
forecasting a future visual field at the requested forecast date based on the output of the trained forecasting model.

11. The method of claim 10, wherein forecasting the future visual field at the requested forecast date based on the output of the trained forecasting model comprises the steps of:
normalizing the most recent VF test vector;
converting and normalizing the most recent date of acquisition to generate a most recent time displacement;
converting and normalizing the requested future date to generate a future time displacement;
normalizing the one or more prior VF test vectors;
converting and normalizing the prior dates of acquisition to generate a set of prior time displacements;
providing the one or more normalized prior VF test vectors and set of prior time displacements, along with the normalized most recent VF test vector and the most recent time displacement as input to a temporal processing module to generate a set of intermediate temporal representation vectors;
reshaping each of the set of intermediate temporal representation vectors, the normalized most recent VF test vector, the most recent time displacement, and the future time displacement into matrix format, and concatenating them into channels to form an input tensor; and
providing the input tensor as input to a trained 2-D convolutional neural network (2-D CNN), thereby generating the future visual field at the requested forecast date.

12. The method of claim 11, wherein the 2-D CNN comprises a hybrid convolution and transformer architecture including an inverted residual convolution layer, a relative self-attention block, and a fully connected layer.

13. The method of claim 11, wherein the temporal processing module comprises a recurrent neural network (RNN) followed by a fully connected layer and a Gaussian Error Linear Unit (GeLU) activation function.

14. The method of claim 13, wherein the RNN is implemented using a long short-term memory (LSTM) architecture.

15. The method of claim 13, wherein the RNN is implemented using a gated recurrent unit (GRU) architecture.
